(19) United States
(12) Patent Application Publication
(10) Pub. No.: US 2026/0041837 A1
(43) Pub. Date: Feb. 12, 2026

(54) ENDOLUMINAL TREATMENT DEVICES AND RELATED METHODS

(71) Applicant: Boston Scientific Scimed, Inc., Maple Grove, MN (US)

(72) Inventor: John Thomas FAVREAU, Charlton, MA (US)

(73) Assignee: Boston Scientific Scimed, Inc., Maple Grove, MN (US)

(21) Appl. No.: 19/291,142

(22) Filed: Aug. 5, 2025

Related U.S. Application Data
(60) Provisional application No. 63/679,874, filed on Aug. 6, 2024.

(51) Int. Cl. A61M 1/00

(52) U.S. Cl. CPC A61M 1/916; A61M 1/918; A61M 1/94; A61M 2205/0216; A61M 2205/3331

(57) ABSTRACT

Medical devices and methods for endoscopic medical procedures, such as closing a wound or otherwise treating tissue, are described herein. The medical device may include a tube having a distal end and a balloon at the distal end. The balloon may include a collapsed configuration and an expanded configuration. The tube may include an inflation lumen and a suction lumen. The balloon may include an inner layer, an outer layer, a first volume, a second volume, and one or more supports positioned within the second volume. The outer layer may surround the inner layer and may define a plurality of pores through the outer layer. The first volume may be defined within the inner layer and is in fluid communication with the inflation lumen. The second volume may be defined between the inner layer and the outer layer and is in fluid communication with the suction lumen.

### CROSS-REFERENCE TO RELATED APPLICATION(S)

This application claims the benefit of priority to U.S. Provisional Application No. 63/679,874, filed on Aug. 6, 2024, which is incorporated by reference herein in its entirety.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Patients with perforations, post-surgical leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices of the current disclosure may rectify some of the deficiencies described above or address other aspects of the art.

### SUMMARY

Each of the aspects disclosed herein may relate to devices and methods for endoluminal wound treatment and may include one or more of the features described in connection with any of the other disclosed aspects.

The disclosure includes an exemplary medical device comprising a tube and a balloon. The tube includes an inflation lumen and a suction lumen. The balloon is positioned at a distal end of the tube. The balloon includes an inner layer and an outer layer surrounding the inner layer and defines a plurality of pores through the outer layer. The balloon further includes a first volume, defined within the inner layer and in fluid communication with the inflation lumen, and a second volume, defined between the inner layer and the outer layer and in fluid communication with the suction lumen. The balloon also includes one or more supports positioned within the second volume. The balloon includes a collapsed configuration and an expanded configuration.

In some examples, the medical device may include any and all of the following features. A first end of each of the one or more supports may contact an outer surface of the inner layer and a second end of each of the one or more supports may contact an inner surface of the outer layer. Each of the one or more supports may be cylindrical. The one or more supports may include a sponge that may fill at least a portion of the second volume. In the collapsed configuration, the first volume may be smaller than the first volume in the expanded configuration. The second volume may be approximately the same volume in the collapsed configuration and the expanded configuration. A shape of each of the one or more supports may be the same in the collapsed configuration and the expanded configuration. The one or more supports may include a porous material. The inner layer may include an elastic material. The first volume may be fluidly isolated from the second volume. The first volume may be configured to receive a fluid from the inflation lumen. The balloon may be configuration to transition from the collapsed configuration to the expanded configuration as the first volume receives the fluid. The second volume may be configured to receive a negative pressure from the suction lumen, in the collapsed configuration and the expanded configuration. The plurality of pores may be configured to provide negative pressure at a target site.

In one or more examples, the medical device may include any and all of the following features. The one or more supports may include one or more walls. Each of the one or more walls may define a plurality of pores through the wall. Each of the one or more walls may be sinusoidal or wavy. The one or more walls may circumscribe one or more portions of the inner layer.

The disclosure also includes another exemplary medical device comprising a tube and a balloon. The tube having a distal end and comprises an inflation lumen and a suction lumen. The balloon is positioned at the distal end of the tube. The balloon includes a first wall, enclosing an inner volume, in fluid communication with the inflation lumen, and a second wall, completely surrounding the first wall. The balloon further includes one or more supports extending from an outer surface of the first wall to an inner surface of the second wall. The second wall including a porous material. The balloon includes a collapsed configuration and an expanded configuration.

In some examples, the medical device may include any and all of the following features. The first wall and the second wall may include an elastic, expandable material. The one or more supports may be configured to maintain a distance between the first wall and the second wall. The second wall may include a plurality of pores in fluid communication with the suction lumen.

The disclosure includes a medical method comprising moving a balloon to a target site. The balloon including an inner wall and an outer wall. The outer wall is porous, contains the inner wall, and is at a distal end of a tube. The inner wall surrounds a first volume. The inner wall and the outer wall define a second volume. The medical method further comprises, via inflation fluid from the tube, transitioning the balloon from a first configuration, in which the inner wall is collapsed, to a second configuration, in which the inner wall is expanded and the outer wall contacts the target site. The medical method comprises applying a negative pressure to the second volume using the tube such that a second fluid at the target site passes form an outer surface of the outer wall into the second volume.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1A depicts an exemplary medical device in an inflated configuration.

FIG. 1B depicts the medical device of FIG. 1A in a collapsed configuration.

FIG. 2 depicts a perspective view of a portion of the medical device of FIGS. 1A-1B.

FIG. 3 depicts a perspective view of a portion of another exemplary medical device.

FIG. 4 depicts a perspective view of a portion of another exemplary medical device.

FIGS. 5A-5D depict the distal end of the medical device of FIGS. 1A-2 within a target site in the collapsed configuration in (FIG. 5A), in the inflated configuration (FIG. 5B), and in partially collapsed configurations (FIGS. 5C and 5D).

### DETAILED DESCRIPTION OF THE FIGURES

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. The term “diameter” also includes widths of elements that are not circular or do not have a circular cross-section.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Endoluminal vacuum therapy (EVT or EVAC, as referred to herein) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is typically delivered to the wound site in the GI tract, for example, through a nasogastric tube having structure at its distal end to apply suction to the site. A proximal end of the tube may be connected to a collection container. The distal structure is typically placed endoscopically into the perforation, leak, or other wound.

In some examples, EVAC includes endoluminal placement of a foam or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., colon). Negative pressure then is applied.

The distal structure at the end of the nasogastric tube in the wound, along with the negative pressure, may accelerate healing by encouraging local tissue granulation at a wound site. When a medical professional utilizes a tube with a sponge/foam at the distal end, the foam may be replaced with increasing smaller sizes of foam as the wound heals and closes. Devices and systems suited for EVAC are limited. For example, EVAC typically requires a foam to be replaced every 3 to 5 days to reduce the risk of tissue ingrowth. Furthermore, infection of the wound may occur, which may prolong treatment of the wound. It should be understood that the terms “sponge” and “foam” as used in this disclosure are interchangeable with one another and refer to the same structure.

Presently, physicians manually shape (e.g., cut) a sponge, such as a Granufoam™ sponge, and manually suture it to a vacuum tube (e.g., a nasogastric tube). Such a process may introduce multiple sources of uncertainty for a physician, particularly if the physician is less experienced in performing EVAC. The physician may be unsure of how large or small to make the sponge to accommodate a target cavity/site, and the physician may be unsure of how deep within the target cavity/site to place the sponge.

Aspects of this disclosure include devices and methods that incorporate a balloon at the distal end of an EVAC device, the balloon having variable shapes and sizes. The balloon may include an inner volume defined by a first layer and an outer volume defined between the first layer and a second layer. The balloon may be attached to a distal end of a tube having one or more lumens for applying negative pressure and inflating/deflating the balloon. The balloon may include one or more rigid or semi-rigid members extending from the first layer to the second layer, the members configured to maintain the outer volume and/or a distance between the first layer and the second layer with the balloon being inflated or collapsed. The second layer may include a plurality of holes, pores, or perforations (referred to interchangeably throughout this disclosure) through a surface of the second layer. The plurality of pores may be in fluid communication to the outer volume and the suction lumen. Other balloons may include specific shapes, such as rounded distal ends or pointed distal ends, to achieve desired patient outcomes. Any features of the balloons discussed within this disclosure may be combined in any suitable manner.

Reference will now be made in detail to examples of the present disclosure described above and illustrated in the accompanying drawings. Wherever possible, reference numbers with the same two terminating digits (e.g., 1XX, 3XX, 4XX) will be used throughout the drawings to refer to the same or like parts.

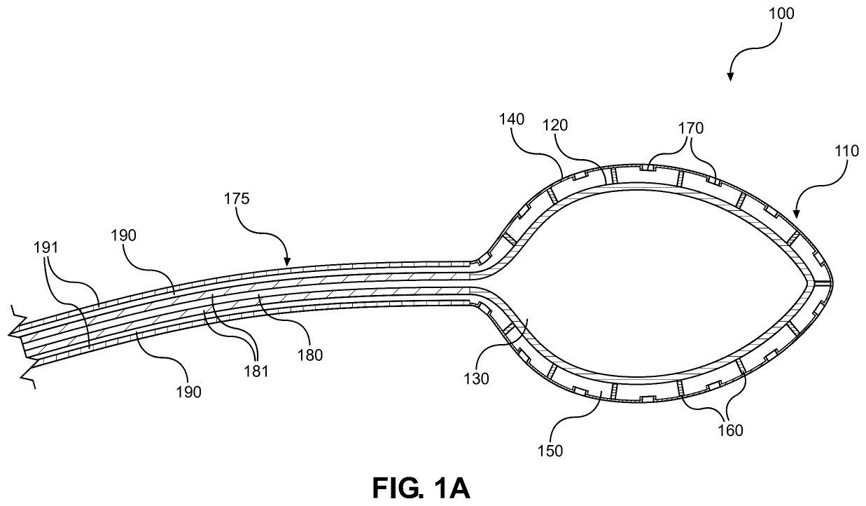

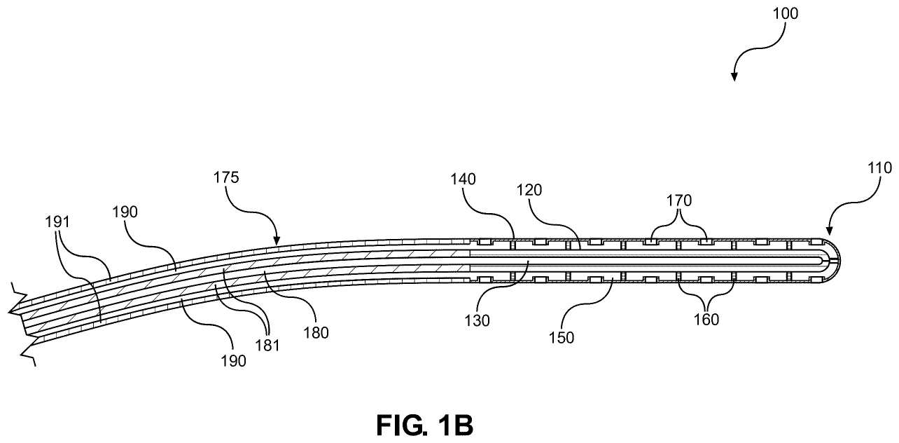

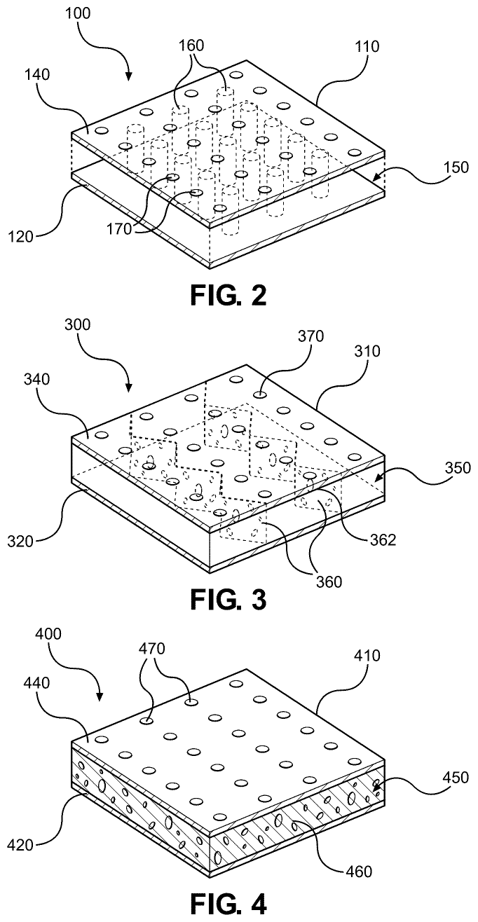

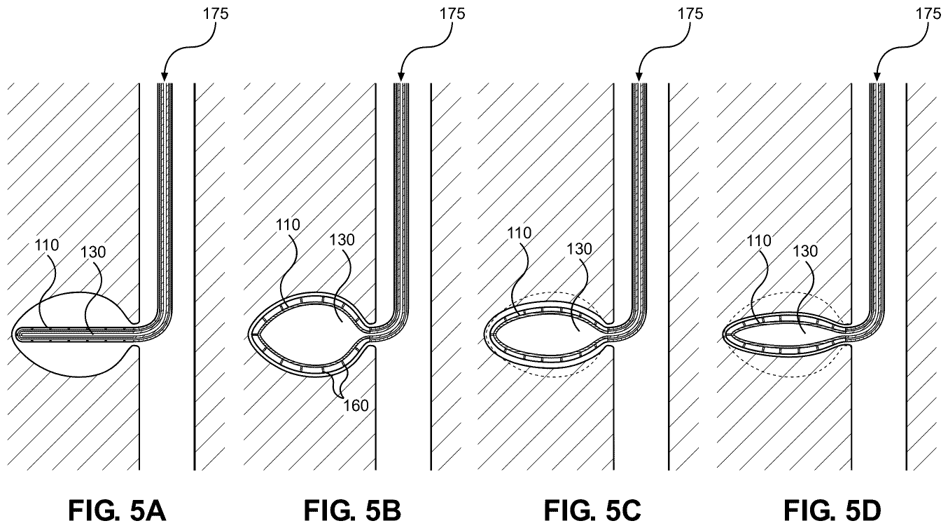

FIGS. 1A-1B and 2 depict a medical device 100 in accordance with an example of this disclosure. In examples, device 100 may be utilized to perform an EVAC procedure. Device 100 may be inserted into a patient for treatment of wounds using negative pressure via a vacuum (as shown in FIGS. 5A-5D, described further herein). Device 100 includes a balloon 110 with a first, inner layer 120 and a second, outer layer 140, and a tube 175 configured to deliver fluid and provide negative pressure to balloon 110. Tube 175 may be configured to transition balloon 110 between an inflated/expanded configuration, a deflated/collapsed configuration, and a plurality of partially collapsed configurations between the inflated and collapsed configurations.

Balloon 110 may be attached to a distal end of tube 175. Balloon 110 may be sized and shaped to be inserted into a target site (e.g., a perforation or wound in a wall of a bodily lumen or cavity). As mentioned above, balloon 110 includes the first, inner layer or inner wall 120 (herein “first layer 120”) and the second, outer layer or outer wall 140 (herein “second layer 140”). Second layer 140 may radially surround at least a portion of first layer 120, and in embodiments, second layer 140 fully encloses first layer 120. First layer 120 may define and/or enclose an inner volume 130. Tube 175 may include an inflation lumen 180 in fluid communication with inner volume 130. First layer 120 may include an elastic and/or expandable material and may be configured to be inflated/expanded and deflated/collapsed via fluid through a distal end of inflation lumen 180. A proximal end of inflation lumen 180 may be connected to a fluid source.

First layer 120 and second layer 140 may define an outer volume 150 between first layer 120 and second layer 140. In other words, first layer 120 and second layer 140 may define outer volume 150 between an exterior surface of first layer 120 and an inner surface of second layer 140. Tube 175 may include a suction lumen 190 in fluid communication with outer volume 150.

As mentioned above, first layer 120 of balloon 110 may include an ellipsoidal shape in the expanded/inflated configuration; however this is merely exemplary, and first layer 120 may include any suitable shape. As discussed above, first layer 120 may include an elastic and/or flexible material. First layer 120 may be formed from one or more of silicone, PEBAX (polyether block amide), nylon, polyurethane, thermoplastic elastomer and other copolymers, and combinations thereof. Accordingly, as fluid is delivered to inner volume 130 via inflation lumen 180, one or more dimensions of first layer 120 may expand and inner volume 130 may increase in volume. Further, as fluid is removed from inner volume 130 via inflation lumen 180, one or more dimensions of first layer 120 may retract and inner volume 130 may decrease in volume. Balloon 110 may be in the inflated configuration when inner volume 130 is sufficiently inflated so that the exterior surface of second layer 140 contacts at least a portion of the target site (see e.g., FIG. 5B) and/or balloon 110 substantially occupies/fills a volume of the target site. Balloon 110 may be in the collapsed configuration when an insufficient amount of fluid is within inner volume 130 such that one or more dimensions of first layer 120 are not expanded (see e.g., FIGS. 1B and 5A). Balloon 110 may be in a partially inflated configuration when inner volume 130 includes less fluid than in the fully-inflated configuration (see e.g., FIGS. 5C and 5D).

Second layer 140 may include a shape corresponding to the shape of first layer 120. For example, second layer 140 may include an ellipsoidal shape with a larger volume than first layer 120, however this is merely exemplary and second layer 140 may include any shape. Second layer 140 may be sized and shaped to enclose, completely or partially, first layer 120.

Second layer 140 includes a porous material with a plurality of pores, holes, perforations and/or channels 170. As shown in FIGS. 1A-1B, each of the plurality of pores 170 extends through the inner surface and exterior surface of second layer 140. Plurality of pores 170 may be in fluid communication with outer volume 150 such that plurality of pores 170 may provide negative pressure at the exterior surface of second layer 140 when negative pressure is applied to outer volume 150. Second layer 140 may include an elastic and/or expandable material. Second layer 140 may be formed from one or more of silicone, PEBAX (polyether block amide), nylon, polyurethane, thermoplastic elastomer and other copolymers, and combinations thereof. Accordingly, as fluid is delivered to inner volume 130, one or more dimensions of second layer 140 may expand. Further, as fluid is removed from inner volume 130, one or more dimensions of second layer 140 may collapse/retract. According to some aspects of the disclosure, one or more of plurality of pores 170 may be interconnected with one or more other pores of plurality of pores 170.

Inner volume 130 may be filled with fluid/fluid may be removed from inner volume 130 to increase/decrease one or more dimensions of inner volume 130 and outer volume 150. For example, filling inner volume 130 or removing fluid from inner volume 130 may permit controlled radial/diametric expansion of balloon 110 (e.g., radial/diametric expansion of balloon 110 relative to a lateral cross section of balloon 110). Higher fluid pressure or greater amounts of fluid within inner volume 130 may correspond to greater diameters of inner volume 130 and outer volume 150. For example, when inner volume 130 includes greater amounts of fluid or higher fluid pressure, balloon 110 may be in the expanded configuration or a partially expanded/partially deflated configuration. Moreover, lower fluid pressure or lesser (or negligible) amounts of fluid within inner volume 130 may correspond to lesser diameters of inner volume 130 and outer volume 150. For example, balloon 110 may be in the collapsed or a partially deflated configuration when inner volume 130 includes less amounts of fluid or lesser fluid pressure. During use, the user may inflate inner volume 130 with a sufficient amount of fluid so that a diameter of balloon 110 is approximately equal to a diameter or dimension of a wound at the target site.

As shown in FIGS. 1A and 1B, tube 175 may include inflation lumen 180 and suction lumen 190. Suction lumen 190 may be defined by a volume enclosed by suction wall 191 and radially outside of inflation wall 181. Similarly, inflation lumen 180 may be defined by a volume enclosed by inflation wall 181. Suction wall 191 may annularly/radially surround inflation wall 181 along a portion or entirety of a length of tube 175. For example, inflation lumen 180 and suction lumen 190 may extend from a proximal end of tube 175 to a distal end of tube 175. Inflation lumen 180 may be fluidly isolated from suction lumen 190 by inflation wall 181. Inflation lumen 180 and/or inflation wall 181 may be coaxial or approximately coaxial with suction lumen 190 and/or suction wall 191 along at least a portion of the length of tube 175.

Inflation lumen 180 may be in fluid communication with inner volume 130, and fluid may flow distally through inflation lumen 180 to inflate inner volume 130 or proximally through inflation lumen 180 to collapse inner volume 130. Similarly, suction lumen 190 may be in fluid communication with outer volume 150 and fluid may flow proximally from outer volume 150 through suction lumen 190. Suction lumen 190 may be configured to provide negative pressure to outer volume 150. A proximal end of suction lumen 190 may be connected to a vacuum source. Suction lumen 190 and outer volume 150 may be fluidly isolated from inflation lumen 180 and inner volume 130.

Balloon 110 may include one or more members 160 (also called “support members” or “supports”) disposed within the outer volume 150. One or more members 160 may extend from the inner surface of second layer 140 to the outer surface of first layer 120. One or more members 160 may be linear and extend radially outward from the outer surface of first layer 120. Members 160 provide structural support between layers 120, 140 so that outer volume 150 is maintained in expanded configurations and collapsed configurations of balloon 110.

Although FIGS. 1A-1B depict a cross sectional view of medical device 100, it should be understood that one or more members 160 may extend outward from first layer 120 at any position about the outer surface of first layer 120. In other words, when viewing FIGS. 1A-1B, it should be understood that one or more members 160 that extend at any angle from first layer 120 toward and away from the viewer have been omitted, among other omitted features, to highlight other features of medical device 100. For example, FIG. 2 depicts a perspective view of a portion of balloon 110 where one or more members 160 extend from a plurality positions on first layer 120. One or more members 160 may include a rigid material or semi-rigid material, such as nylon, PEBAX, polyurethane, silicone, other materials with similar properties, materials known by those skilled in the art, and combinations thereof. One or more members 160 may be configured to maintain their size and shape in the inflated configuration, the collapsed configuration, and partially collapsed configurations of balloon 110. For example, a length of one or more members 160 and a distance between first layer 120 and second layer 140 may be maintained in the inflated configuration, the collapsed configuration and transitory configurations between a fully inflated configuration and a fully collapsed configuration. In other words, the length of one or more members 160 and the distance between first layer 120 and second layer 140 may remain the same in the inflated, collapsed, and transitory configurations. As shown in FIGS. 1A-2, one or more members 160 may include a cylindrical shape. However one or more members 160 are not limited to including a cylindrical shape and may include any shape such as, but not limited to, prisms (e.g., rectangular, hexagonal, etc.) or any other three-dimensional shape. According to some aspects of the disclosure, a sponge may fill a portion or an entirety of second volume 150 surrounding one or more members 160. The sponge may be in fluid communication with plurality of pores 170 such that fluid from the target site may be siphoned through pores 170, the sponge, and second volume 150, and through the suction lumen 180. The sponge may be formed from GranuFoam™, silicone, PVA (polyvinyl alcohol), polyurethane, EPDM (Ethylene-propylene diene monomer), and/or other material with similar properties or known by those skilled in the art

In some examples, when transitioning between configurations, an end of one or more members 160 connected to second layer 140 and/or an end of one or more members 160 connected to first layer 120 may pivot relative to the respective connected layer 120, 140, allowing member 160 to lay flat or relatively flat against layers 120, 140, lessening the distance between first layer 120 and second layer 140, to make a fully collapsed configuration of balloon 110 have a lesser profile. Such a configuration is especially suitable for delivery to the treatment site.

FIGS. 5A-5D depict an exemplary medical device (e.g., medical device 100) being used during an exemplary medical procedure (e.g., an EVAC procedure). Prior to beginning the procedure, the user may select a balloon (e.g., balloon 110) with appropriate collapsed and expandable dimensions to be inserted into the target site. The user may select an appropriate size and shape of balloon 110 based on the size, shape, and location of the target site and so that balloon 110 is insertable into the target site for use. After selecting the balloon, the user may navigate (e.g., move) balloon 110 and tube 175 to the target site and then may insert balloon 110 into the target site, using any suitable delivery method, as shown in FIG. 5A. Any suitable delivery method may include, but is not limited to, insertion through a working channel of an endoscope. It should be understood that balloon 110 is inserted into the target site in the collapsed configuration (FIGS. 1B, 5A).

As seen in FIG. 5B, after inserting balloon 110 into the target site, the user may inflate the inner volume 130 with a fluid (e.g., a gas or liquid) via inflation lumen 180 of tube 175. Balloon 110 may be inflated until it transitions to the inflated configuration. For example, the user may activate or actuate the fluid source to pump fluid through inflation lumen 180 into inner volume 130 until balloon 110 expands to fill or partially fill the volume of the target site, or an exterior surface of second layer 140 contacts an interior surface of the target site (see FIG. 5B). One or more members 160 may be configured to maintain a distance between first layer 120 and second layer 140

After the user has transitioned balloon 110 to the inflated configuration, the user may begin providing negative pressure to outer volume 150 via suction lumen 190 of tube 175. For example, the user may activate or actuate the vacuum source to provide negative pressure to suction lumen 190, outer volume 150, and through plurality of pores 170 of second layer 140. As negative pressure is being provided to outer volume 150, fluid at the target site may be siphoned through plurality of pores 170 into outer volume 150 and through the suction lumen 190. For example, the vacuum source or other receptacle may be fluidly connected to a proximal end of tube 175 and/or suction lumen 190 and may be configured to receive fluid from the target site.

As seen in FIG. 5C, after the passage of three to five days (or other suitable time period), the wound at the target site may begin to heal and shrink. To allow the wound to continue to heal and shrink, the user may partially deflate balloon 110 by removing some of the fluid from inner volume 130 to reduce the volume of balloon 110. For example, the fluid source may activated or actuated to remove fluid from inner volume 130 via inflation lumen 180. After and/or during partial deflation, negative pressure may continue to be applied to outer volume 150. The user may repeat the above steps (e.g., waiting a predetermined period of time, partially deflating inner volume 130, and continuing to apply negative pressure to outer volume 150) as the wound continues to heal and shrink until balloon 110 has transitioned to the collapsed configuration (see FIGS. 1B, 5D) and/or until the target site has sufficiently healed to no longer require fluid removal at the target site. After balloon 110 has transitioned to the collapsed configuration, the user may continue to apply negative pressure to outer volume 150 until the user has determined that the wound has sufficiently healed and/or no longer needs negative pressure to be applied to remove fluid from the target site. Then the user may remove balloon 110 and tube 175 from the target site/bodily lumen of the patient by any suitable removal method.

FIG. 3 depicts another a portion of another exemplary medical device 300 based on aspects of the disclosure. Medical device 300 and its respective components may include any of the features of medical device 100, and may be used in the exemplary methods described above in the same way. As noted above, wherever possible, reference numbers with the same two terminating digits (e.g., 1XX, 3XX, 4XX) will be used throughout the disclosure to refer to the same or like parts. As compared with medical device 100, aspects of medical device 300 may have different components for maintaining a distance between the first layer and the second layer of the balloon. Medical device 400 may be used in an exemplary procedure (e.g., an EVAC procedure) in the same or similar manner as medical device 100 as shown in FIGS. 5A-5D. Medical device 300 may include a balloon 310 attached (e.g., coupled) to a distal end of a tube (e.g., tube 175). Balloon 310 may include a first layer 320 and a second layer 340. First layer 320 and second layer 340 may define an outer volume 350 between an exterior surface of first layer 320 and an interior surface of second layer 340. Second layer 340 may be formed from a porous material and may include a plurality of pores 370 in fluid communication with outer volume 350 and a suction lumen.

Balloon 310 may include one or more members 360 (e.g., one or more walls 360) extending from the exterior surface of first layer 320 to the interior surface of second layer 340. One or more walls 360 may extend along a perimeter or circumference of first layer 320. For example, one or more walls 360 may extend along a perimeter or circumference of first layer 320 at one or more lateral cross sections of balloon 310. According to an aspect in which first layer 320 includes an ellipsoidal shape, one or more walls 360 may circumscribe first layer 320 at one or more lateral cross sections of balloon 310. Walls 360 may be at any angle to the longitudinal axis of balloon 310, for example, parallel or perpendicular to the longitudinal axis, or any other suitable angle. One or more walls 360 may include a rigid or semi-rigid material (such as those mentioned above for members 160) and may maintain the distance between first layer 320 and second layer 340 when balloon 310 is in the inflated configuration or collapsed configuration, or as balloon 310 transitions between the inflated configuration and the collapsed configuration.

One or more walls 360 may be a porous material and may include a plurality of pores 362. Plurality of pores 362 may extend from a first side of one or more walls 360 to a second side of one or more walls 360, opposite to the first side. For example, plurality of pores 362 may extend from a proximal side of one or more walls 360 to a distal side of one or more walls 360. Alternatively, pores 362 may be randomly distributed throughout wall 360, like pores within a sponge. Plurality of pores 362 may permit fluid siphoned into outer volume 350 from the target site to pass through walls 360 toward the suction lumen (e.g., suction lumen 190).

As seen in FIG. 3, each wall 360 may include a zigzag shape as it extends through outer volume 350. However, the zigzag-shape is merely exemplary and walls 360 may include any shape such as a wavy-shape, a sinusoidal-shape, linear-shape, and other shapes. In some examples, one of one or more walls 360 may include a first shape and another of one or more walls 360 may include a second shape, different from the first shape. Each wall 360 may be flexible and/or elastic to change shapes as balloon 310 inflates and maintain connections with layers 320, 340, yet be sufficiently rigid to maintain outer volume 350 in expanded configurations. It should be understood that balloon 310 may include one or more members 160 in addition to one or more walls 360.

FIG. 4 depicts another exemplary medical device 400 based on aspects of the disclosure. Medical device 400 and its respective components may include any of the features of medical device 100, 300. As noted above, wherever possible, reference numbers with the same two terminating digits (e.g., 1XX, 3XX, 4XX) will be used throughout the disclosure to refer to the same or like parts. As compared with medical device 100, aspects of medical device 400 may have different components for maintaining a distance between the first layer and the second layer of the balloon. Medical device 400 may be used in an exemplary procedure (e.g., an EVAC procedure) in the same or similar manner as medical device 100 as shown in FIGS. 5A-5D. Medical device 400 may include a balloon 410 attached (e.g., coupled) to a distal end of a tube (e.g., tube 175). Balloon 410 may include a first layer 420 and a second layer 440. First layer 420 and second layer 440 may define an outer volume 450 between an exterior surface of first layer 420 and an interior surface of second layer 440. Second layer 440 may be formed from a porous material and may include a plurality of pores 470 in fluid communication with outer volume 450 and a suction lumen.

Balloon 410 may include one or more members 460 (e.g., one or more sponges 460) extending from the exterior surface of first layer 420 to the interior surface of second layer 440. For example, one or more sponges 460 may include one sponge 460 that occupies all, substantially all, or at least a portion of outer volume 450. In some embodiments, one or more sponges 460 may include two or more sponges 460 that together occupies all, substantially all, or at least a portion of outer volume 450. It should be understood that sponge 460 may be formed from GranuFoam^{TM} or other similar materials. For example, sponge 460 may be formed from silicone, PVA (polyvinyl alcohol), polyurethane, EPDM (Ethylene-propylene diene monomer), and/or other material with similar properties or known by those skilled in the art. Sponge 460 may include sufficiently rigid material so that sponge 460 may maintain a distance between first layer 420 and second layer 440 when balloon 410 is in the inflated configuration, collapsed configuration, and transitory configurations between the inflated configuration and the collapsed configuration. When negative pressure is applied to outer volume 450 via suction lumen (e.g., suction lumen 190), fluid from the target site may be siphoned through plurality of pores 470 into outer volume 450 and sponge 460, and then may be pulled through sponge 460 (e.g., pulled through a plurality of pores of sponge 460) and outer volume 450 toward the suction lumen. It should be understood that balloon 410 may also include one or more members 160 and/or one or more walls 360. In embodiments with one or more walls 360, one or more sponges 460 may be a number of sponges corresponding to a number of walls 360.

According to some aspects of the disclosure, second layer 440 may be omitted and instead an outermost surface of balloon 110 may be an exterior surface of sponge 460. For example, referring to FIG. 4, second layer 440 may be omitted. Fluid from the target site may be pulled through a plurality of pores at the exterior surface of sponge 460 and be siphoned through outer volume 450 and/or suction lumen 190.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a tube having a distal end, the tube comprising:
an inflation lumen, and
a suction lumen; and
a balloon at the distal end, the balloon including:
an inner layer,
an outer layer surrounding the inner layer and defining a plurality of pores through the outer layer,
a first volume defined within the inner layer and in fluid communication with the inflation lumen,
a second volume defined between the inner layer and the outer layer and in fluid communication with the suction lumen, and
one or more supports positioned within the second volume,
wherein the balloon includes a collapsed configuration and an expanded configuration.

2. The medical device of claim 1, wherein a first end of each of the one or more supports contacts an outer surface of the inner layer, and a second end of each of the one or more supports contacts an inner surface of the outer layer.

3. The medical device of claim 2, wherein each of the one or more supports is cylindrical.

4. The medical device of claim 2, wherein the one or more supports include one or more walls, wherein each of the one or more walls defines a plurality of pores through the wall.

5. The medical device of claim 4, wherein each of the one or more walls is sinusoidal or wavy.

6. The medical device of claim 4, wherein the one or more walls circumscribe one or more portions of the inner layer.

7. The medical device of claim 1, wherein the one or more supports includes a sponge, wherein the sponge fills at least a portion of the second volume.

8. The medical device of claim 1, wherein, in the collapsed configuration, the first volume is smaller than the first volume in the expanded configuration.

9. The medical device of claim 8, wherein the second volume is approximately a same volume in the collapsed configuration and the expanded configuration.

10. The medical device of claim 1, wherein a shape of each of the one or more supports is the same in the collapsed configuration and the expanded configuration.

11. The medical device of claim 1, wherein the one or more supports include a porous material.

12. The medical device of claim 1, wherein the inner layer includes an elastic material.

13. The medical device of claim 1, wherein the first volume is fluidly isolated from the second volume.

14. The medical device of claim 1, wherein the first volume is configured to receive a fluid from the inflation lumen, wherein the balloon is configured to transition from the collapsed configuration to the expanded configuration as the first volume receives the fluid.

15. The medical device of claim 14, wherein the second volume is configured to receive a negative pressure from the suction lumen, in the collapsed configuration and the expanded configuration, wherein the plurality of pores is configured to provide negative pressure at a target site.

16. A medical device comprising:
a tube having a distal end, the tube comprising:
an inflation lumen, and
a suction lumen; and
a balloon positioned at the distal end including:
a first wall enclosing an inner volume in fluid communication with the inflation lumen,
a second wall completely surrounding the first wall;
one or more supports extending from an outer surface of the first wall to an inner surface of the second wall;
wherein the second wall includes a porous material;
wherein the balloon includes a collapsed configuration and an expanded configuration.

17. The medical device of claim 16, wherein the first wall and the second wall include an elastic, expandable material.

18. The medical device of claim 16, wherein the one or more supports are configured to maintain a distance between the first wall and the second wall.

19. The medical device of claim 16, wherein the second wall includes a plurality of pores in fluid communication with the suction lumen.

20. A medical method comprising:
moving a balloon to a target site, the balloon including an inner wall and an outer wall, wherein the outer wall is porous, contains the inner wall, and is at a distal end of a tube, wherein the inner wall surrounds a first volume, and the inner wall and the outer wall define a second volume;
via inflation fluid from the tube, transitioning the balloon from a first configuration, in which the inner wall is collapsed, to a second configuration, in which the inner wall is expanded, and the outer wall contacts the target site; and
applying a negative pressure to the second volume using the tube, such that a second fluid at the target site passes from an outer surface of the outer wall into the second volume.
